(19) United States
(12) Patent Application Publication
(10) Pub. No.: US 2025/0073464 A1
(43) Pub. Date: Mar. 6, 2025

(54) Device for Neurostimulation

(71) Applicant: PAJUNK GMBH MEDIZINTECHNOLOGIE, Geisingen (DE)

(72) Inventors: Simone PAJUNK-SCHELLING, Geisingen (DE); Martin HAUGER, Donaueschingen (DE)

(73) Assignee: PAJUNK GMBH MEDIZINTECHNOLOGIE, Geisingen (DE)

(21) Appl. No.: 18/821,876

(22) Filed: Aug. 30, 2024

(30) Foreign Application Priority Data
Sep. 5, 2023 (DE) 102023123896.5

(51) Int. Cl. A61N 1/36; A61M 5/34; A61N 1/05

(52) U.S. Cl. CPC A61N 1/36017; A61M 5/347; A61N 1/0502; A61M 2205/02; A61M 2205/054

(57) ABSTRACT

The application relates to a device for neurostimulation with an electrically conductive stimulation wire with a diameter, a distal end and a proximal end, an electrically conductive stimulation electrode disposed at the distal end of the stimulation wire, an electrically conductive stimulation cable connected at the proximal end of the stimulation wire, an electrically insulating tubing with an inner diameter, an outer diameter, a distal end, and a proximal end, and an injection tube slipped in sections over the proximal end of the tubing forming a seal, wherein the stimulation wire is disposed within the tubing, wherein the inner diameter of the tubing is greater than the diameter of the stimulation wire such that an interspace for supplying fluid is formed in the tubing, wherein the stimulation electrode projects beyond the distal end of the tubing.

### CROSS-REFERENCE TO RELATED APPLICATION

The present application claims priority to German Patent Application No. 10 2023 123 896.5, filed Sep. 5, 2023, the entirety of which is incorporated herein by reference.

### FIELD OF APPLICATION

The application relates to a device for neurostimulation.

### BACKGROUND

It is known for the alleviation of pain or for relaxing muscle tensions and contractions to transmit electrical current pulses, for example low-frequency alternating current at low voltage by means of electrodes placed onto the skin in order to stimulate nerves transcutaneously in this manner. However, the skin represents herein a comparatively high contact resistance. As a rule, the stimulation furthermore takes place over a large area.

The application therefore addresses the problem of specifying an improved device for neurostimulation which, in particular, is of versatile use and with which preferably a more direct stimulation is feasible.

### SUMMARY

The problem of the application is resolved through a device for neurostimulation with the characteristics of patent claim 1.

Advantageous embodiments and further developments of the application are specified in the dependent claims.

The device for neurostimulation according to the application comprises an electrically conductive stimulation wire with a diameter, a distal end and a proximal end, which is disposed in an electrically insulating tubing with an inner diameter, an outer diameter, a distal end and a proximal end, wherein the inner diameter of the tubing is greater than the diameter of the stimulation wire such that an interspace for the supply of fluid is formed in the tubing, wherein on the distal end of the stimulation wire a stimulation electrode is electrically conductive disposed, which projects beyond the distal end of the tubing, and wherein the proximal end of the stimulation wire projects beyond the proximal end of the tubing, and, bent around the proximal end of the tubing, is guided to the outer side of the tubing, wherein an electrically conductive stimulation cable is connected, such that it is electrically conductive, to the proximal end of the stimulation wire and wherein an injection tube is slipped in sections over the proximal end of the tubing to form a sealing.

The device according to the application is suited for neurostimulation as well as also for the supply of a fluid, for example of a fluid such as an anesthetic agent. By disposing the stimulation wire within the lumen of the tubing is obtained, according to the application, an especially compact structure. The stimulation wire, bent around at the proximal end of the tubing, enables the simple linkage of the stimulation cable on the outer side of the tubing without having to guide the stimulation wire through the wall of the tubing such that a possible leakage site can be avoided.

Due to the flexibility of the tubing, moreover, an elongated flexible device can be provided which can be introduced into the body either through cannulae or through indwelling cannulae but can also be introduced into the body through natural body orifices, for example through the nose, the bladder or the vagina without the necessity of having to make an incision in the skin. Thereby also the stimulation of nerves can be carried out which are only accessible with difficulty or not all at all with stimulation electrodes that are attached to the skin outside of the body.

According to an advantageous further development of the application, the stimulation electrode comprises a rounded outer surface and, for example, is developed dome-shaped, spherical, ellipsoidal, mushroom-shaped, pear-shaped, or egg-shaped. Due to the rounded physical form, injuries on the body of the person to be treated can be avoided. Moreover, such physical form of the stimulation electrode can simplify advancing the device, in particular into the body orifice.

A preferred embodiment of the application provides for the stimulation electrode to have an outer diameter that is, at least in cross section, greater than the outer diameter of the tubing. Stated differently, at least the greatest outer diameter of the stimulation electrode is greater than the outer diameter of the tubing. Due to such an implementation as large a surface of the stimulation electrode as is feasible can be provided which can improve the stimulation response of the stimulation electrode.

It is fundamentally conceivable that fluid can exit at the distal end of the tubing between the stimulation electrode and the tubing wall. The stimulation electrode is advantageously disposed under form closure with a securement section in the distal end of the tubing, wherein the tubing comprises in its wall at least one injection opening terminating into the interspace between the stimulation wire and the tubing and/or the stimulation electrode comprises an axial through-opening. The form-closure disposition enables the stabilization of the distal end of the stimulation wire within the tubing. The disposition under form closure can furthermore enable a closure of the distal end of the tubing in order to prevent the penetration of particles into the distal end of the tubing or even prevent clogging of the distal end of the tubing. In this embodiment the fluid can exit laterally through the at least one injection opening disposed in the wall of the tubing and/or through the axial through-opening of the stimulation electrode.

The proximal end of the tubing with the connection site to the stimulation cable and the connection site to the injection tube, are preferably disposed in a housing, wherein the stimulation cable and the injection tube exit proximally, in particular parallel to the longitudinal axis of the tubing, from the housing. The proximal end of the tubing including the connection sites can herein be inlaid into the housing and additionally be encast with an adhesive agent or be ensheathed with an injection molded housing. In this way the connection sites can be disposed under protection.

The diameter of the stimulation wire is preferably less than 0.3 mm, preferably approximately 0.2 mm. In this way a thin flexible stimulation wire can be provided which can enable the simple advance into the body, in particular into natural body orifices.

The stimulation wire is preferably fabricated of special steel. The requisite stability of the stimulation wire can thereby be achieved, especially also at lengths of more than 10 cm.

According to an especially preferred embodiment of the application, the outer diameter of the tubing is less than 1.0 mm, preferably less than 0.9 mm. The dimensioning of the device for neurostimulation can thereby be enabled which can enable the simple introduction into the body, in particular into natural body orifices.

The inner diameter of the tubing is preferably in the range of 0.4 mm to 0.7 mm, preferably in the range of 0.5 mm to 0.6 mm. Such dimensioning enables a sufficiently large interspace between the inner wall of the tubing and the stimulation wire in order to be able to guide a fluid through the tubing from the proximal to the distal end.

According to a preferred further development of the application, the length of the tubing is more than 10 cm, preferably more than 15 cm, especially preferred more than 20 cm, for example 32.2 cm. Thereby a sufficient length is provided in order to be able to advance the device for neurostimulation sufficiently far into the body, for example into natural body orifices.

A preferred embodiment of the application provides for the tubing to be fabricated of a synthetic material, for example of a polyamide. Such material can enable sufficient impermeability, high flexibility as well as good biocompatibility.

The tubing preferably comprises markings, starting at the distal end, which can visualize for a user by which length the tubing has already been advanced into the body,

A device according to the application is utilized according to the application for neurostimulation through a natural body orifice, for example the nose, the bladder or the vagina. For example, when introduced through the nose, the nerve stimulation of the sphenopalatine ganglion or also, by injection, a blockage of the sphenopalatine ganglion can be carried out in order to be able to treat, for example, facial and head pain in the case of migraine attacks.

An embodiment example of the application will be explained in detail in conjunction with the following Figures. Therein depict

### BRIEF DESCRIPTION OF THE DRAWINGS

FIG. 1 a side view of an embodiment example of a device for neurostimulation according to the application,

FIG. 2 a detailed view of the distal end of the device according to FIG. 1,

FIG. 3 a longitudinal section of the distal end according to FIG. 2,

FIG. 4 a partially sectioned view of the device according to FIG. 1, and

FIG. 5 an enlargement of a cutout of FIG. 4.

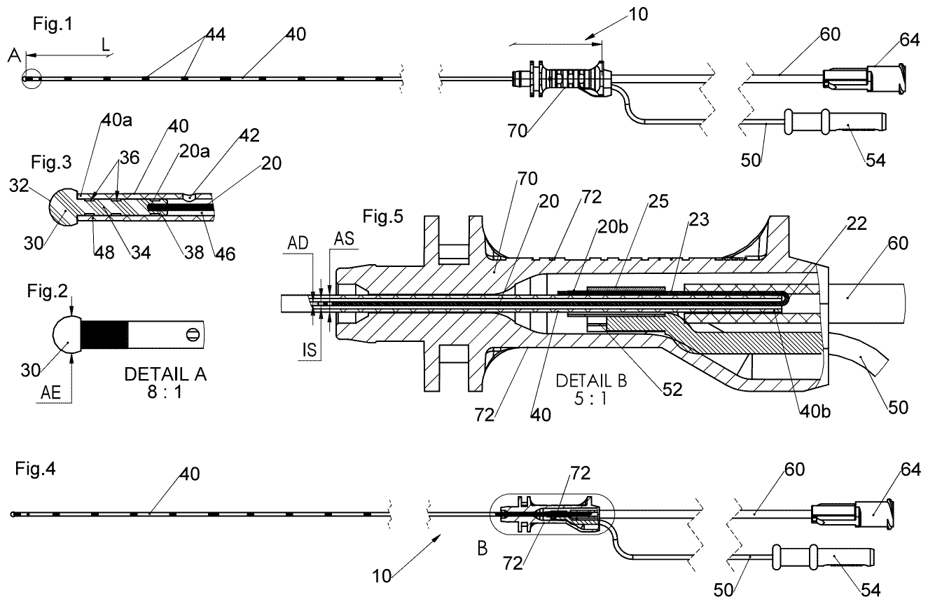

FIGS. 1 to 5 show various views of an embodiment example of a device 10 for neurostimulation. For the sake of greater clarity, not all reference symbols are specified in all Figures.

### DETAILED DESCRIPTION

The device 10 comprises an electrically conductive stimulation wire 20 with a diameter AD, a distal end 20a and a proximal end 20b. The stimulation wire 20 involves in particular a flexible, bendable wire. The stimulation wire 20 is fabricated, for example, of special steel. The diameter AD of the stimulation wire 20 can be less than 0.3 mm, preferably approximately 0.2 mm.

The device 10 furthermore comprises an electrically insulating tubing 40 having an inner diameter IS, an outer diameter AS, a distal end 40a and a proximal end 40b. The tubing 40 involves in particular a flexible, impermeable and biocompatible tubing 40. The tubing 40 can be fabricated, for example, of a synthetic material, preferably a polyamide. The outer diameter AS of the tubing 40 is preferably less than 1.0 mm, especially preferably less than 0.9 mm and can be, for example, 0.87 mm. The inner diameter IS of the tubing 40 is greater than the diameter AD of the stimulation wire 20 and can be in the range of 0.4 mm to 0.7 mm, preferably in the range of 0.5 mm to 0.6 mm and be, for example, 0.54 mm. The length L of the tubing 40 is more than 10 cm, preferably more than 15 cm, especially preferably more than 20 cm, for example 32.2 cm. The tubing 40 can comprise markings 44 starting at the distal end 40a (see FIGS. 1 and 4) which, in particular, are disposed equidistantly and can provide information to the user regarding the length of the distal end 40a of tubing 40 by which the tubing 40 has been advanced into a body.

The stimulation wire 20 is disposed in tubing 40, in particular guided through the lumen of tubing 40. Thereby that the inner diameter IS of tubing 40 is greater than the diameter AD of the stimulation wire 20 an interspace 46 is formed in tubing 40, in particular in the manner of an annular interspace between the stimulation wire 20 and the inner wall of tubing 40 through which, as will be described in greater detail in the following, fluid can be conducted from the proximal end 40b of tubing 40 to the distal end 40a of tubing 40.

At the distal end 20a of the stimulation wire 20 a stimulation electrode 30 is disposed, such that it is electrically conductive, which projects beyond the distal end 40a of tubing 40. The stimulation electrode 30 can have a rounded outer surface 32 and be developed, for example, dome-shaped, spherical, ellipsoidal, mushroom-shaped, pear-shaped or egg-shaped or in any other spheroidally rounded form. Due to the convex physical form of the stimulation electrode 30, it has, at least in cross section, a greatest outer diameter AE. This outer diameter AE of the stimulation electrode 30 is greater than the outer diameter AS of tubing 40 (cf. in particular FIGS. 2 and 3).

The electrically conductive connection between the stimulation wire 20 and the stimulation electrode 30 can be formed, for example, thereby that the distal end 20a of the stimulation wire 20 is inserted into a stud hole 38 disposed in the stimulation electrode 30 or in a securement section 34 disposed in the stimulation electrode 30 and here fixed under clamping force or fixed by welding (see FIG. 3).

The stimulation electrode 30 can be disposed with the securement section 34 under form closure in the distal end 40a of tubing, or stated more precisely, in a section 48 starting at the distal end 40a of tubing 40. The securement section 34 can comprise one or more grooves 36 disposed on its outer side which improve fixation under clamping force of the securement section 34 in section 48 of the tubing 40. The stimulation electrode 30, in particular the securement section 34, can thereby close off the distal end 40a of tubing 40. In order, nevertheless, to enable a fluid disposed in interspace 46 to exit at the distal end 20a of device 10, tubing 40 can comprise in its wall at least one injection opening 42 (cf. FIG. 3) terminating into the interspace 46 between the stimulation wire 20 and the tubing 40, and/or the stimulation electrode 30 can comprise an axial through-opening (not shown).

The proximal end 20b of the stimulation wire 20 projects beyond the proximal end 40b of tubing 40 and bent, in particular in a bent-around section 22, around the proximal end 40b of the tubing 40, in particular by approximately 180°, is guided to the outer side of tubing 40. An electrically conductive stimulation cable 50, in particular an electrically conductive lead 52 of the stimulation cable 50, is connected, in particular such that it is electrically conductive, at the proximal end 20b of the stimulation wire 20 which, in particular with a carried-back section 23, adjoining the bent-around section 22, comes to lie in contact on the outer side of tubing 40. The electrically conductive contacting of the stimulation wire 20 can thus take place outside of the lumen, which when in use is often filled with fluid, of tubing 40 and without perforation through the wall of the tubing 40. The stimulation cable 50, in particular the lead 52 of the stimulation cable 50, can be connected, for example, electrically conductive by means of a crimp sleeve 25 with the stimulation wire 20, in particular with the carried-back section 23. As depicted in FIGS. 1 and 4, at the other end of the stimulation cable 50 an electrical plug-in connector 54 can be disposed for the connection of a control unit with a pulse generator.

An injection tube 60 is slipped in sections over the proximal end 40b of tubing 40, and therewith, in particular, also in sections over the carried-back section 23 in contact on the outer side of tubing 40 of the stimulation wire 20, forming a sealing. This can be achieved for example thereby that an inner diameter of the injection tube 60 corresponds approximately to the outer diameter AS of tubing 40. As depicted in FIGS. 1 and 4, at the other end of injection tube 60 an injection connection 64 can be disposed across which, for example using a syringe, a fluid, for example an anesthetic agent, can be injected into the injection tube 60 which across the injection tube 60 enters into the proximal end 40b of tubing 40 and leaves it at the distal end 40a of tubing 40, in particular through the injection opening 42.

The proximal end 40b of tubing 40, with its connection site to the stimulation cable 50 and the connection site to the injection tube 60, can be disposed in a housing 70, wherein the stimulation cable 50 and the injection tube 60 exit from the housing 70 proximally, in particular parallel to the longitudinal axis of tubing 40. The proximal end 40b of tubing 40, including the connection sites, can herein be inlaid into the housing 70 and additionally be encast with an adhesive agent or, alternatively, be ensheathed with an injection molded housing 70. In this way, the connection sites can be disposed such that they are protected. On the housing 70 one or more recessed grips 72 can be disposed for placement of a user's fingers in order to increase the grip certainty.

A device 10 can be utilized for neurostimulation through a natural body orifice, for example the nose, the bladder or the vagina. For example, when inserted through the nose the neurostimulation of the sphenopalatine ganglion can be carried out with the stimulation electrode 30 when applying for example low-frequency alternating current to the stimulation wire 20 or also by injecting an anesthetic agent through the injection tube 60 and tubing 40, blocking of the sphenopalatine ganglion can also be carried out in order, for example, to be able to treat facial and head pain in cases of migraine attacks.

### LIST OF REFERENCE SYMBOLS

10 Device
20 Stimulation wire
20a Distal end of the stimulation wire
20b Proximal end of the stimulation wire
22 Bent-around section
23 Carried-back section
25 Crimp sleeve
30 Stimulation electrode
32 Outer surface of the stimulation electrode
34 Securement section
36 Groove
38 Blind or stud hole
40 Tubing
40a Distal end of the tubing
40b Proximal end of the tubing
42 Injection opening
44 Marking
46 Interspace
48 Section
50 Stimulation cable
52 Lead
54 Electric plug-in connector
60 Injection tube
64 Injection connection
70 Housing
72 Recessed grips
AD Diameter of stimulation wire
AS Outer diameter of the tubing
IS Inner diameter of the tubing
AE Outer diameter of stimulation electrode
L Length of the tubing

What is claimed:

1. A device for neurostimulation comprising:
an electrically conductive stimulation wire with a diameter, a distal end and a proximal end,
an electrically conductive stimulation electrode disposed at the distal end of the stimulation wire,
an electrically conductive stimulation cable connected at the proximal end of the stimulation wire,
an electrically insulating tubing with an inner diameter, an outer diameter, a distal end, and a proximal end, and
an injection tube slipped in sections over the proximal end of the tubing forming a seal,
wherein the stimulation wire is disposed within the tubing,
wherein the inner diameter of the tubing is greater than the diameter of the stimulation wire such that an interspace for supplying fluid is formed in the tubing,
wherein the stimulation electrode projects beyond the distal end of the tubing,
wherein the proximal end of the stimulation wire projects beyond the proximal end of the tubing and bends around the proximal end of the tubing such that the proximal end of the stimulation wire is guided to an outer side of the tubing.

2. The device of claim 1, wherein the stimulation electrode has a rounded outer surface and is dome-shaped, spherical, ellipsoidal, mushroom-shaped, pear-shaped or egg-shaped.

3. The device of claim 1, wherein the stimulation electrode at least in a cross section has an outer diameter which is greater than the outer diameter of the tubing.

4. The device of claim 1, wherein the stimulation electrode further comprises a securement section disposed on the distal end by the stimulation electrode, wherein the securement section is disposed within the tubing, wherein the tubing further comprises at least one injection opening in a wall of the tubing terminating into the interspace between stimulation wire and tubing and/or the stimulation electrode comprises an axial through-opening.

5. The device of claim 1, wherein the proximal end of the tubing further includes a connection site to the stimulation cable and a connection site to the injection tube, wherein the connection sites are disposed in a housing, wherein the stimulation cable and the injection tube are led proximally out of the housing parallel to the longitudinal axis of the tubing.

6. The device of claim 1, wherein the diameter of the stimulation wire is less than 0.3 mm.

7. The device of claim 1, wherein the stimulation wire is fabricated of special steel.

8. The device of claim 1, wherein the outer diameter of the tubing is less than 1.0 mm.

9. The device of claim 1, wherein the inner diameter of the tubing is in the range of 0.4 mm to 0.7 mm.

10. The device of claim 1, wherein the length of the tubing is more than 10 cm.

11. The device of claim 1, wherein the tubing is fabricated of a synthetic material.

12. The device of claim 1, wherein the tubing comprises markings starting at the distal end.

13. A device for neurostimulation comprising:
an electrically insulating tubing with a tubing distal end and a tubing proximal end;
an electrically conductive stimulation wire located within the tubing and having a wire distal end and a wire proximal end;
an electrically conductive stimulation electrode at the wire proximal end, wherein the electrically conductive stimulation electrode projects beyond the tubing distal end; and
an interspace for supplying fluid between an inner wall of the tubing and an outer surface of the stimulation wire.

14. The device of claim 13, wherein the wire proximal end projects beyond the tubing proximal end and bends around the tubing proximal end such that the wire proximal end is guided to an outer side of the tubing.

15. The device of claim 13, wherein the stimulation electrode further comprises a securement section formed by a distal end of the stimulation electrode, wherein the securement section is disposed with the tubing, wherein the tubing further comprises an injection opening in a wall of the tubing terminating into the interspace between stimulation wire and tubing and/or the stimulation electrode comprises an axial through-opening.
